(19) United States
(12) Patent Application Publication
(10) Pub. No.: US 2025/0018226 A1
(43) Pub. Date: Jan. 16, 2025

(54) NEUTRON CAPTURE THERAPY APPARATUS

(71) Applicant: SUMITOMO HEAVY INDUSTRIES, LTD., Tokyo (JP)

(72) Inventor: Kenzo SASAI, Niihama-shi (JP)

(21) Appl. No.: 18/900,511

(22) Filed: Sep. 27, 2024

Related U.S. Application Data
(63) Continuation of application No. PCT/JP2023/012535, filed on Mar. 28, 2023.

(30) Foreign Application Priority Data
Mar. 31, 2022 (JP) 2022-059071

(51) Int. Cl. A61N 5/10

(52) U.S. Cl. CPC A61N 5/1078; A61N 5/1045; A61N 2005/109; A61N 2005/1092

(57) ABSTRACT

A neutron capture therapy apparatus includes a treatment unit including an irradiator irradiating an irradiation target with a neutron ray, a simulation unit simulating position adjustment of the target with respect to the irradiator in the treatment unit, a first moving mechanism capable of moving the target in the treatment unit, a second moving mechanism capable of moving the target with respect to the simulation unit, and a transport mechanism capable of transporting the target from the second moving mechanism to the first moving mechanism, in which the second moving mechanism moves the target such that a target position with respect to the simulation unit is substantially the same as a predetermined target position with respect to the irradiator in the treatment unit, and the first moving mechanism moves the target to the predetermined target position with respect to the irradiator in the treatment unit.

### CROSS-REFERENCE TO RELATED APPLICATIONS

This is a bypass continuation of International PCT Application No. PCT/JP2023/012535, filed on Mar. 28, 2023, which claims priority to Japanese Patent Application No. 2022-059071, filed on Mar. 31, 2022, which are incorporated by reference herein in their entirety.

### BACKGROUND

### Technical Field

A certain embodiment of the present disclosure relates to a neutron capture therapy apparatus.

### Description of Related Art

Boron neutron capture therapy (BNCT) using a boron compound is known as a neutron capture therapy that irradiates a cancer cell with a neutron ray to kill the cancer cell. In the boron neutron capture therapy, boron that has been previously incorporated into the cancer cell is irradiated with the neutron ray to selectively destroy the cancer cell using scattering of a heavy charged particle generated by the irradiation.

For example, a neutron capture therapy apparatus disclosed in the related art is known as the neutron capture therapy apparatus as described above. In the neutron capture therapy apparatus disclosed in the related art, a patient is disposed in front of an irradiation outlet of a neutron ray in a state where the patient is fixed to a mounting member to perform treatment. For this reason, positioning of the patient is performed in a preparation room, and the positioning is performed again after the patient is transported to a treatment room.

### SUMMARY

According to an embodiment of the present invention, there is provided a neutron capture therapy apparatus according to the present disclosure including a treatment unit including an irradiator that irradiates an irradiation target with a neutron ray, a simulation unit that simulates adjustment of a position of the irradiation target with respect to the irradiator in the treatment unit, a first moving mechanism that is capable of moving the irradiation target in the treatment unit, a second moving mechanism that is capable of moving the irradiation target with respect to the simulation unit, and a transport mechanism that is capable of transporting the irradiation target from the second moving mechanism to the first moving mechanism, in which the second moving mechanism moves the irradiation target such that a position of the irradiation target with respect to the simulation unit is substantially the same as a predetermined position of the irradiation target with respect to the irradiator in the treatment unit, and the first moving mechanism moves the irradiation target to the predetermined position of the irradiation target with respect to the irradiator in the treatment unit.

### BRIEF DESCRIPTION OF THE DRAWINGS

FIG. 1 is a schematic view of a neutron capture therapy apparatus according to an embodiment of the present disclosure.

FIG. 2 is a schematic view of a configuration near a treatment unit of the neutron capture therapy apparatus.

FIG. 3 is a schematic plan view of a first moving mechanism of a treatment room.

FIG. 4 is a schematic plan view of a second moving mechanism of a preparation room.

FIG. 5 is a schematic plan view of a state where a transport mechanism transports a patient.

FIG. 6 is a side surface view of the transport mechanism.

FIG. 7 is a schematic plan view of a state where positioning of the patient is performed in the preparation room.

FIG. 8 is a schematic plan view of a state where the positioning of the patient is performed in the preparation room.

FIG. 9 is a schematic plan view of a state where positioning of the patient is performed in the treatment room.

FIG. 10 is a schematic plan view of a state where the positioning of the patient is performed in the treatment room.

### DETAILED DESCRIPTION

In the above neutron capture therapy apparatus, the positioning is performed by a positioning mechanism in the preparation room, the positioning mechanism and the irradiation target are transported to the treatment room by a transport mechanism, and the positioning is performed again by the positioning mechanism in the treatment room. In such a neutron capture therapy apparatus, due to the influence of a positional error in a traveling drive or the like of the transport mechanism, a deviation occurs between a position of the positioning mechanism in the treatment room and a position of the positioning mechanism in the treatment room. Accordingly, there is an issue in that positioning accuracy of the irradiation target during the irradiation is lowered.

Therefore, it is desirable to provide a neutron capture therapy apparatus capable of improving positioning accuracy of an irradiation target during irradiation.

In the neutron capture therapy apparatus according to the present disclosure, the treatment unit includes the first moving mechanism that is capable of moving the irradiation target, the second moving mechanism that is capable of moving the irradiation target with respect to the simulation unit, and the transport mechanism that is capable of transporting the irradiation target from the second moving mechanism to the first moving mechanism. Accordingly, in a case where the positioning with respect to the simulation unit using the second moving mechanism is completed, the transport mechanism transports the irradiation target to the treatment unit. The first moving mechanism moves the irradiation target such that the positioning in the simulation unit is reproduced. The second moving mechanism moves the irradiation target such that the position of the irradiation target with respect to the simulation unit is substantially the same as the predetermined position of the irradiation target with respect to the irradiator in the treatment unit, and the first moving mechanism moves the irradiation target to the predetermined position of the irradiation target with respect to the irradiator in the treatment unit. In this case, the first moving mechanism in the treatment unit can accurately reproduce the state of the positioning with respect to the simulation unit using the second moving mechanism. From the above, it is possible to improve the positioning accuracy of the irradiation target during the irradiation.

The irradiator of the treatment unit may include a first collimator, and the simulation unit may include a second collimator that simulates the first collimator. In this case, it is possible to simulate the positioning of the irradiation target with respect to the simulation unit in consideration of even a position of the collimator.

The neutron capture therapy apparatus may further include a storage unit, in which the storage unit may store a parameter of the second moving mechanism at a time of positioning with respect to the simulation unit, and the first moving mechanism may perform positioning of the irradiation target based on the parameter stored in the storage unit. In this case, in the treatment unit, the first moving mechanism can easily and accurately reproduce the state of the positioning with respect to the simulation unit using the second moving mechanism.

The neutron capture therapy apparatus may further include a fixation portion that fixes the irradiation target, in which the first moving mechanism and the second moving mechanism may move the fixation portion to move the irradiation target, and the transport mechanism may transport the irradiation target together with the fixation portion. In this case, a posture of the irradiation target in a case where the positioning thereof is performed with respect to the simulation unit is kept constant from the transport by the transport mechanism to the positioning by the first moving mechanism. Therefore, in the treatment unit, the first moving mechanism can accurately reproduce the state of the positioning with respect to the simulation unit using the second moving mechanism.

According to the present disclosure, it is possible to provide the neutron capture therapy apparatus capable of improving the positioning accuracy of the irradiation target during the irradiation.

Hereinafter, a preferred embodiment of the present disclosure will be described in detail with reference to drawings.

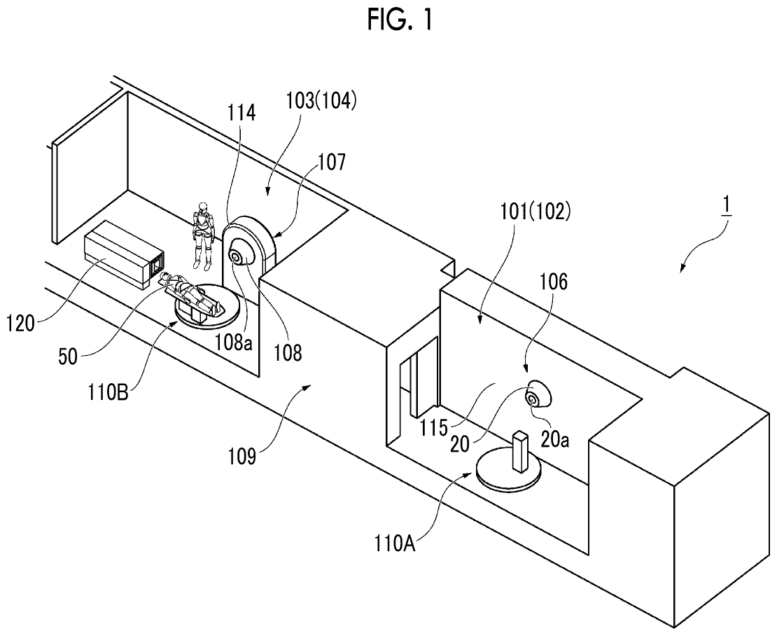

FIG. 1 is a schematic view of a neutron capture therapy apparatus 1 according to an embodiment of the present disclosure. The neutron capture therapy apparatus 1 performs cancer treatment using a boron neutron capture therapy (BNCT). The neutron capture therapy apparatus 1 includes a treatment unit 102, a preparation unit 104 including a simulation irradiation port 107 (simulation unit), a first moving mechanism 110A, a second moving mechanism 110B, and a transport mechanism 120.

The treatment unit 102 has an irradiation port 106 (irradiator) that irradiates a patient 50 with a neutron ray N. The treatment unit 102 is configured by a structure or the like in which the irradiation port 106 and the first moving mechanism 110A are disposed. The treatment unit 102 is provided in a treatment room 101. The irradiation port 106 is provided on a vertical wall portion of the treatment room 101. The neutron ray N exits from the irradiation port 106 in a horizontal direction. The irradiation port 106 includes a collimator 20 and an irradiator periphery wall 115, which will be described below. The first moving mechanism 110A is capable of moving the patient 50 in the treatment unit 102. The first moving mechanism 110A is provided at a position in front of the irradiation port 106 in the treatment room 101.

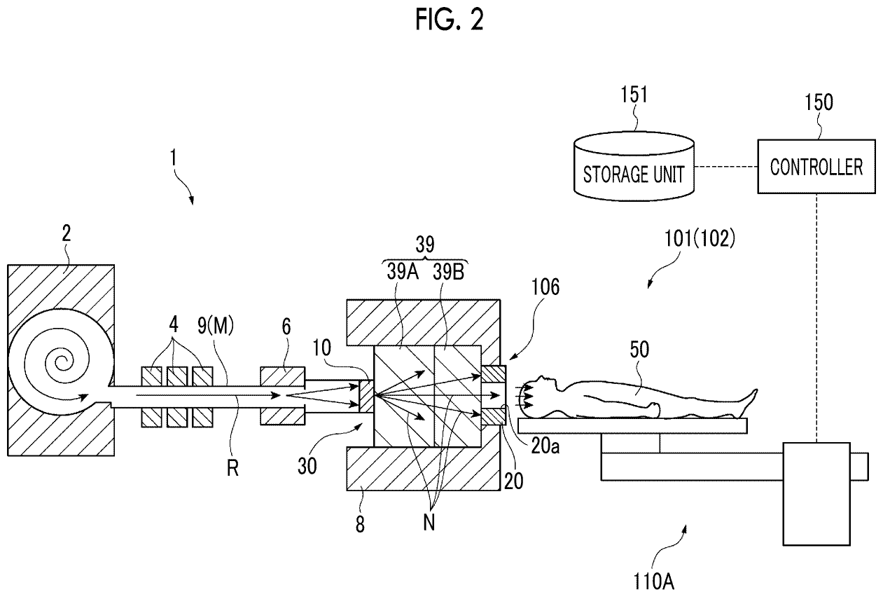

A configuration around the treatment unit 102 will be described with reference to FIG. 2. In the treatment unit 102, for example, a tumor of the patient 50 (irradiation target) to whom boron (10B) is administered is irradiated with the neutron ray N.

The neutron capture therapy apparatus 1 includes an accelerator 2. The accelerator 2 accelerates a particle, and exits a particle beam R. For example, a cyclotron, a linear accelerator, or the like may be employed as the accelerator 2.

The particle beam R exited from the accelerator 2 passes through a transport path 9, which is referred to as a beam duct whose inside is kept vacuum and that allows a beam to pass through, to be transported to a target disposition portion 30. The target disposition portion 30 is to dispose a target 10, and has a mechanism that holds the target 10 to be in a posture during the irradiation. The target disposition portion 30 disposes the target 10 at a position facing an end portion (exit outlet) of the transport path 9. The particle beam R exited from the accelerator 2 passes through the transport path 9, and advances toward the target 10 disposed at the end portion of the transport path 9. A plurality of electromagnets 4 (quadrupole electromagnets or the like) and a scanning electromagnet 6 are provided along the transport path 9. The plurality of electromagnets 4 performs, for example, beam axis adjustment of the particle beam R using the electromagnet.

The scanning electromagnet 6 scans the particle beam R to perform irradiation control of the particle beam R for the target 10. The scanning electromagnet 6 controls an irradiation position of the particle beam R for the target 10.

In the neutron capture therapy apparatus 1, the target 10 is irradiated with the particle beam R to generate the neutron ray N, and the neutron ray Nis exited toward the patient 50. The neutron capture therapy apparatus 1 includes the target 10, a shield member 8, a deceleration member 39, and the collimator 20.

The target 10 receives the irradiation with the particle beam R to generate the neutron ray N. The target 10 is a solid-shaped member made of a material that generates the neutron ray N in a case of being irradiated with the particle beam R. Specifically, the target 10 is made of, for example, beryllium (Be), lithium (Li), tantalum (Ta), or tungsten (W), and has, for example, a disk-shaped solid shape having a diameter of 160 mm. The target 10 is not limited to the disk shape, and may have another shape.

The deceleration member 39 decelerates the neutron ray N generated by the target 10 (decreases energy of the neutron ray N). The deceleration member 39 may have a laminated structure consisting of a layer 39A that mainly decelerates a fast neutron contained in the neutron ray N, and a layer 39B that mainly decelerates an epithermal neutron contained in the neutron ray N.

The shield member 8 shields the generated neutron ray N, a gamma ray generated due to the generation of the neutron ray N, and the like not to be released to the outside. The shield member 8 is provided to surround the deceleration member 39. An upper portion and a lower portion of the shield member 8 extend to an upstream side of the particle beam R from the deceleration member 39.

The first collimator 20 shapes an irradiation field of the neutron ray N and has an irradiation outlet 20a through which the neutron ray N passes. The first collimator 20 is, for example, a block-shaped member having the irradiation outlet 20a at a center thereof. The first collimator 20 is attached to the irradiator periphery wall 115, which is a wall portion of a place where the neutron ray N is irradiated into the treatment room 101.

The preparation unit 104 includes the second moving mechanism 110B and the simulation irradiation port 107 (simulation unit). The simulation irradiation port 107 simulates adjustment of a position of the patient 50 with respect to the irradiation port 106 in the treatment unit 102. The simulation irradiation port 107 has a second collimator 108 that is a simulation collimator that simulates the first collimator 20 of the irradiation port 106, and a simulation wall 114 that simulates a part of the irradiator periphery wall 115 around the irradiation port 106. Further, the simulation irradiation port 107 also has a simulation irradiation outlet 108a, which is a through-hole in the horizontal direction, obtained by simulating the irradiation outlet 20a of the neutron ray N. The second moving mechanism 110B is capable of moving the patient 50 with respect to the simulation irradiation port 107 in the preparation room 103. The second moving mechanism 110B is provided at a position in front of the simulation irradiation port 107 in the preparation room 103.

The transport mechanism 120 is capable of transporting the patient 50 from the second moving mechanism 110B to the first moving mechanism 110A. The transport mechanism 120 is provided in the preparation room 103. A shield area 109 configured such that radiation does not leak to the preparation room 103 is provided between the preparation room 103 and the treatment room 101. In a case where the positioning in the preparation room 103 is completed, the transport mechanism 120 receives the patient 50 from the second moving mechanism 110B. The transport mechanism 120 causes the patient 50 to pass through the shield area 109 to deliver the patient 50 to the first moving mechanism 110A of the treatment room 101.

Further, the neutron capture therapy apparatus 1 includes a controller 150 that controls various devices and a storage unit 151 that stores various types of information used for the control (refer to FIG. 2). The controller 150 controls at least the first moving mechanism 110A, the second moving mechanism 110B, and the transport mechanism 120.

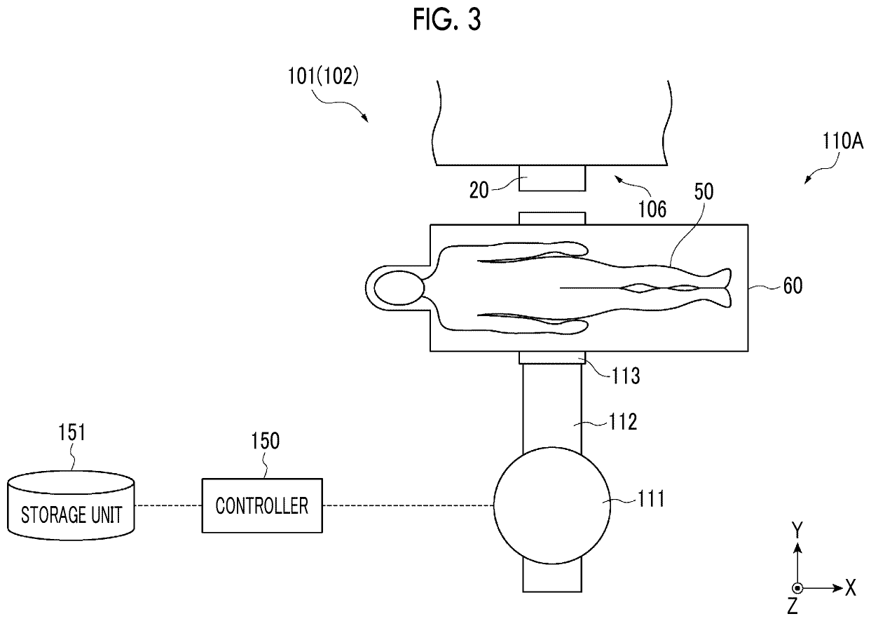

The first moving mechanism 110A will be described in detail with reference to FIG. 3. With setting of an X-axis and a Y-axis in the horizontal direction and a Z-axis in a vertical direction, the description may be made using the XYZ coordinate. The first moving mechanism 110A is a so-called six-axis mechanism that moves the patient 50 in six axial directions. The first moving mechanism 110A is capable of performing horizontal movement in an X-axis direction and rotational movement around the X-axis. The first moving mechanism 110A is capable of performing the horizontal movement in a Y-axis direction and the rotational movement around the Y-axis. The first moving mechanism 110A is capable of performing vertical movement in a Z-axis direction and the rotational movement around the Z axis. In the present embodiment, the first moving mechanism 110A supports the patient 50 in a state of being fixed to a fixation portion 60, and moves the patient 50 together with the fixation portion 60.

The first moving mechanism 110A has a base portion 111 provided on a floor surface at a position separated by a predetermined distance from the irradiation port 106. The base portion 111 is a member extending in an up and down direction, and is rotatable around the Z axis and movable up and down in the Z axis direction. The base portion 111 has an arm portion 112 extending in the horizontal direction. A support portion 113 that supports the fixation portion 60 is provided in a tip part of the arm portion 112. The arm portion 112 can be expanded and contracted such that a position of the support portion 113 is changeable. Further, the arm portion 112 can also adjust an inclination of the support portion 113. The support portion 113 is rotatable around the Z axis with respect to the tip part of the arm portion 112.

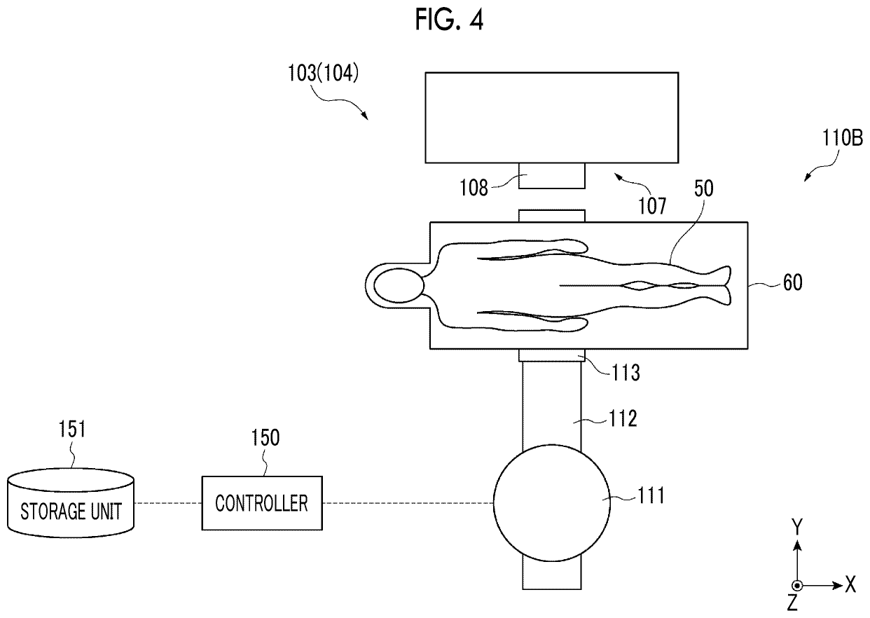

The second moving mechanism 110B will be described with reference to FIG. 4. The second moving mechanism 110B has the same structure as the first moving mechanism 110A. That is, the second moving mechanism 110B has the same base portion 111, arm portion 112, and support portion 113 as the first moving mechanism 110A. A positional relationship between the second moving mechanism 110B and the simulation irradiation port 107 is the same as a positional relationship between the first moving mechanism 110A and the irradiation port 106. A position of the base portion 111 of the second moving mechanism 110B with respect to the second collimator 108 is the same as a position of the base portion 111 of the first moving mechanism 110A with respect to the first collimator 20.

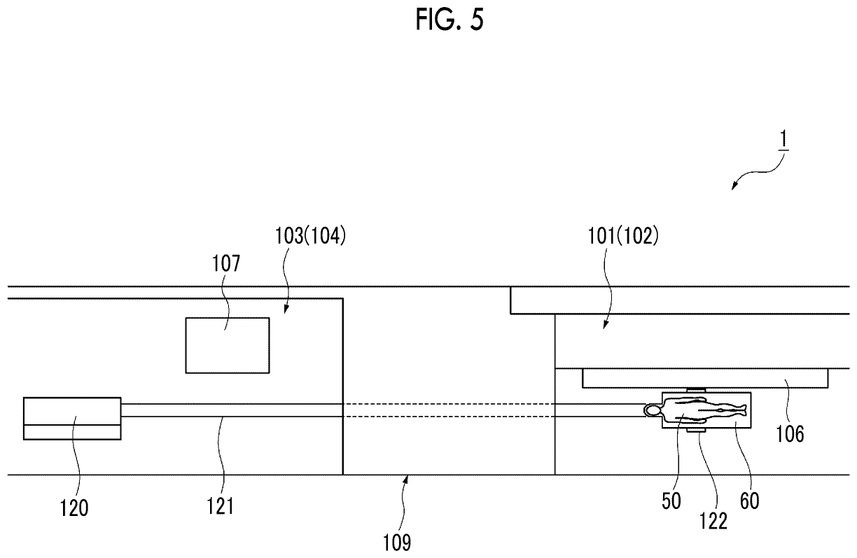

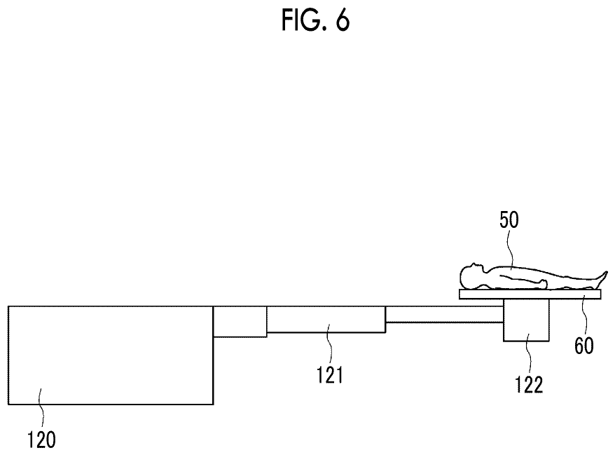

A configuration of the transport mechanism 120 will be described in detail with reference to FIGS. 5 and 6. The transport mechanism 120 includes an arm portion 121 that is expandable and contractible, and a support portion 122 provided at a tip part of the arm portion 121 to support the patient 50 and the fixation portion 60. The transport mechanism 120 supports the fixation portion 60 with the support portion 122 and extends the arm portion 121 in this state to transport the patient 50 from the preparation room 103 to the treatment room 101 via the shield area 109.

In the preparation room 103, the patient 50 is fixed to the fixation portion 60 by using a restraint or the like. The first moving mechanism 110A and the second moving mechanism 110B move the fixation portion 60 to move the patient 50. Further, the transport mechanism 120 transports the patient 50 together with the fixation portion 60.

In the present embodiment, the second moving mechanism 110B moves the patient 50 such that the position of the patient 50 with respect to the simulation irradiation port 107 is substantially the same as a predetermined position of the patient 50 with respect to the irradiation port 106 in the treatment unit 102. The first moving mechanism 110A moves the patient 50 to the predetermined position of the patient 50 (irradiation target) with respect to the irradiation port 106 in the treatment unit 102. The first moving mechanism 110A and the second moving mechanism 110B can respectively move the patient 50 in the treatment unit 102 and the preparation unit 104 such that a positional relationship at a time of positioning between the simulation irradiation port 107 and the patient 50, that is, a positional relationship in a state where the simulation irradiation port 107 and the patient 50 are positioned (positioning is completed) is substantially the same as a positional relationship between the irradiation port 106 and the patient 50 during the irradiation in the treatment unit 102. Here, a range of “substantially the same” is any of the following ranges (1) to (3).

(1) A range of positioning accuracy of a drive mechanism (for example, motor) that drives the first moving mechanism 110A and the second moving mechanism 110B

(2) A range of detection accuracy of positions, which are detected by a position detector such as an imaging device and a sensor, of a site serving as a reference point near an affected part of the patient 50 (for example, a site serving as a reference point for positioning such as a bone, a soft tissue, or a marker drawn on a body surface), the first collimator 20, and the second collimator 108

(3) A range of allowable deviation in treatment

The “predetermined position” is the position of the patient 50 with respect to the irradiation port 106 during the irradiation. The controller 150 controls the first moving mechanism 110A and the second moving mechanism 110B such that the positional relationship at the time of positioning between the simulation irradiation port 107 and the patient 50 is the same as the positional relationship between the irradiation port 106 and the patient 50 during the irradiation in the treatment unit 102. Further, the storage unit 151 stores a parameter (control parameter related to the position of the patient 50) of the second moving mechanism 110B at the time of positioning with respect to the simulation irradiation port 107. The first moving mechanism 110A performs the positioning of the patient 50 based on the parameter stored in the storage unit 151. That is, the controller 150 acquires, from the storage unit 151, the parameter of the second moving mechanism 110B, which is subjected to the positioning. The controller 150 controls the first moving mechanism 110A with the acquired parameter.

Next, a procedure from the positioning in the treatment unit 102 to the irradiation in the treatment unit 102 will be described. Here, so-called multi-division irradiation is assumed to be performed in which the patient 50 is irradiated with the neutron ray N from two directions. Therefore, the patient 50 needs to be positioned in the two directions with respect to the irradiation port 106.

First, as shown in FIG. 4, in the treatment room 101, the fixation portion 60 is attached to the support portion 113 of the second moving mechanism 110B, and the patient 50 is disposed on the fixation portion 60. A worker fixes the patient 50 to the fixation portion 60. At a start of the positioning, the fixation portion 60 is set at a reference position (position shown in FIG. 4).

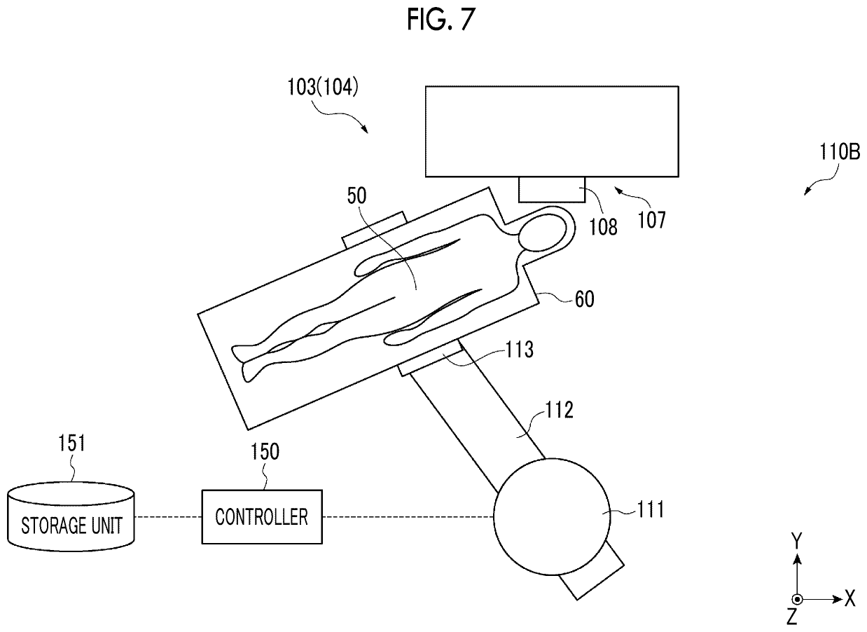

Next, the positioning simulating the irradiation of a first division is performed, as shown in FIG. 7. The worker moves the patient 50 with the second moving mechanism 110B to dispose the affected part near the second collimator 108. In a case where an X-ray device is provided in the preparation unit 104, imaging is performed with X-rays and image collation with a CT image is performed to decide a position correction amount. The second moving mechanism 110B is driven for correction. In a case where only a laser is used for a positioning method, a marker of the patient 50 is positioned in accordance with the laser. In a case where a camera is used for the positioning method, the position of the patient 50 is adjusted according to treatment planning while the marker of the patient 50 is imaged by the camera. In a case where final positioning of the first division is completed, the storage unit 151 stores a positioning position, that is, the parameter of the second moving mechanism 110B at the positioning position.

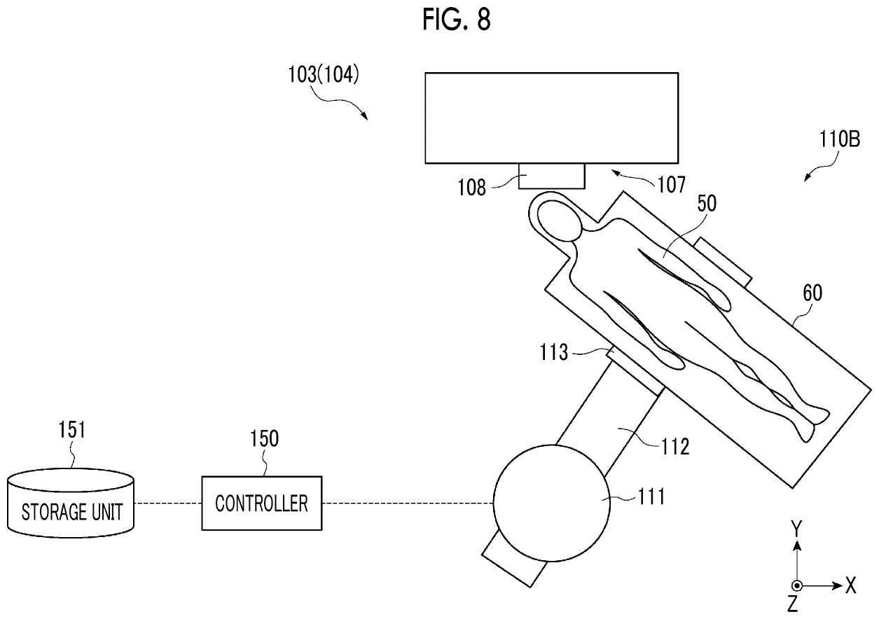

Next, the positioning simulating the irradiation of a second division is performed, as shown in FIG. 8. The worker moves the patient 50 with the second moving mechanism 110B to dispose the affected part near the second collimator 108 in a direction different from a direction of the first division. The positioning is performed by the same positioning method as described above. In a case where final positioning of the second division is completed, the storage unit 151 stores a positioning position, that is, the parameter of the second moving mechanism 110B at the positioning position.

Next, the patient 50 is transferred to the support portion 122 of the transport mechanism 120 together with the fixation portions 60, as shown in FIG. 5. The transport mechanism 120 extends the arm portion 121 to transport the patient 50, together with the fixation portion 60, from the preparation room 103 to the treatment room 101 via the shield area 109.

Next, in a case where the patient 50 arrives at the treatment room 101, the patient 50 is transferred to the support portion 113 of the first moving mechanism 110A together with the fixation portion 60, as shown in FIG. 3. In an initial state, the fixation portion 60 is set at a reference position (position shown in FIG. 3). During the process of transporting the patient 50 by the transport mechanism 120 and of transferring the patient 50 to the first moving mechanism 110A, the patient 50 is continuously fixed to the fixation portion 60, and a deviation of the position of the patient 50 with respect to the fixation portion 60 is prevented from occurring.

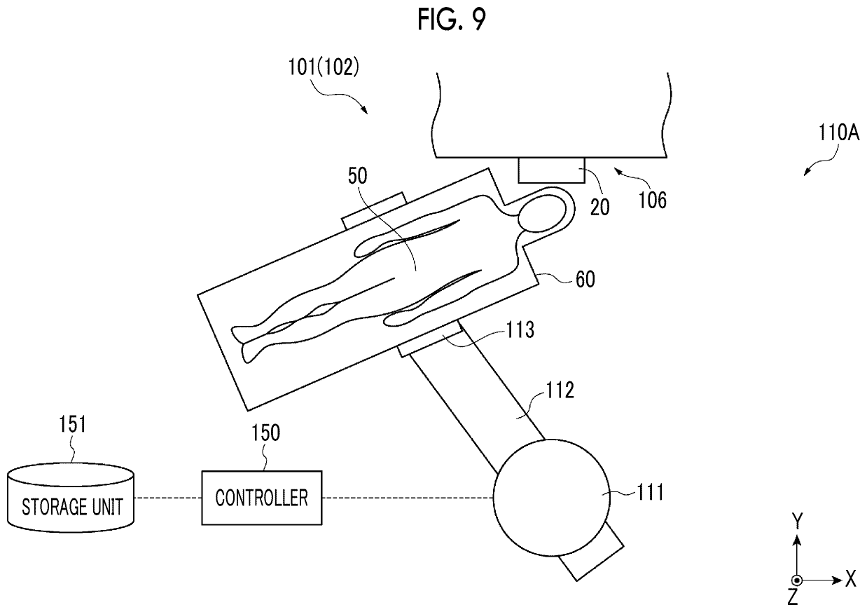

Next, the positioning for performing the irradiation of the first division is performed, as shown in FIG. 9. The controller 150 acquires, from the storage unit 151, the parameter of the second moving mechanism 110B in the positioning of the first division, and the controller 150 controls the first moving mechanism 110A with the parameter. Accordingly, a positional relationship of the first moving mechanism 110A with respect to the first collimator 20 is a positional relationship of the second moving mechanism 110B with respect to the second collimator 108 of the simulation irradiation port 107. That is, a positional relationship of the patient 50 with respect to the first collimator 20 is reproduced as a positional relationship of the patient 50 with respect to the second collimator 108. In a case where the positioning of the patient 50 is completed, the irradiation of the first division is performed.

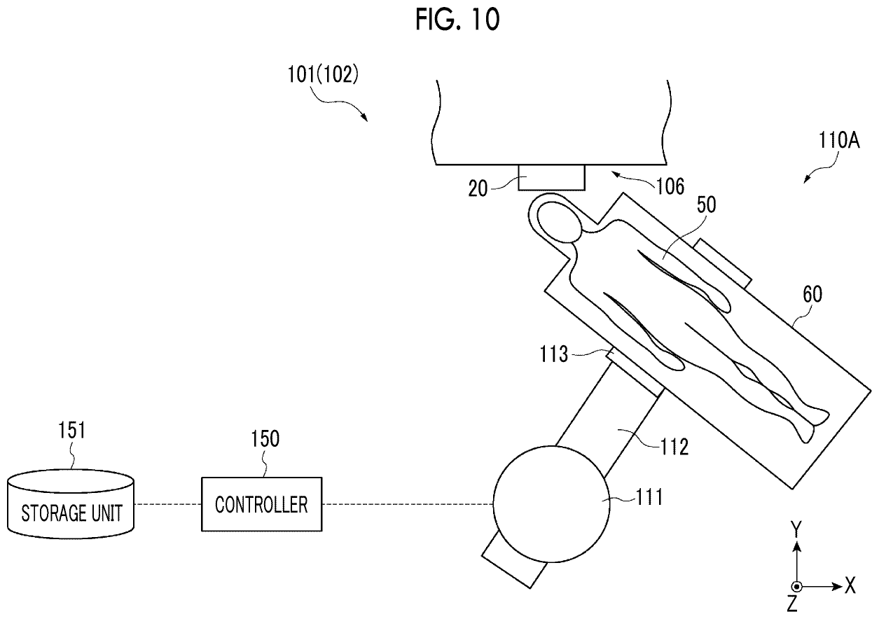

Next, the positioning for performing the irradiation of the second division is performed, as shown in FIG. 10. The controller 150 acquires, from the storage unit 151, the parameter of the second moving mechanism 110B in the positioning of the second division, and the controller 150 controls the first moving mechanism 110A with the parameter. Accordingly, the positional relationship of the first moving mechanism 110A with respect to the first collimator 20 is the positional relationship of the second moving mechanism 110B with respect to the second collimator 108 of the simulation irradiation port 107. That is, the positional relationship of the patient 50 with respect to the first collimator 20 is reproduced as the positional relationship of the patient 50 with respect to the second collimator 108. In a case where the positioning of the patient 50 is completed, the irradiation of the second division is performed.

Next, actions and effects of the neutron capture therapy apparatus 1 according to the present embodiment will be described.

The neutron capture therapy apparatus 1 according to the present embodiment includes the first moving mechanism 110A that is capable of moving the patient 50 in the treatment unit 102, the second moving mechanism 110B that is capable of moving the patient 50 with respect to the simulation irradiation port 107, and the transport mechanism 120 that is capable of transporting the patient 50 from the second moving mechanism 110B to the first moving mechanism 110A. Accordingly, in a case where the positioning with respect to the simulation irradiation port 107 is completed by using the second moving mechanism 110B, the transport mechanism 120 transports the patient 50 to the treatment unit. The first moving mechanism 110A moves the patient 50 such that the positioning with respect to the simulation irradiation port 107 is reproduced. Here, the second moving mechanism 110B moves the patient 50 such that the position of the patient 50 with respect to the simulation irradiation port 107 is substantially the same as the predetermined position of the patient 50 with respect to the irradiation port 106 in the treatment unit 102. The first moving mechanism 110A moves the patient 50 to the predetermined position of the patient 50 with respect to the irradiation port 106 in the treatment unit 102. In this case, in the treatment unit 102, the first moving mechanism 110A can accurately reproduce the state of the positioning with respect to the simulation irradiation port 107 using the second moving mechanism 110B. From the above, it is possible to improve the positioning accuracy of the patient 50 during the irradiation.

The irradiation port 106 of the treatment unit 102 may include the first collimator 20, and the simulation irradiation port 107 may include the second collimator 108 that simulates the first collimator 20. In this case, it is possible to simulate the positioning of the patient 50 with respect to the simulation irradiation port 107 in consideration of even a position of the collimator.

The neutron capture therapy apparatus 1 may further include the storage unit 151. The storage unit 151 may store the parameter of the second moving mechanism 110B at the time of positioning with respect to the simulation irradiation port 107, and the first moving mechanism 110A may perform the positioning of the patient 50 based on the parameter stored in the storage unit 151. In this case, in the treatment unit 102, the first moving mechanism 110A can easily and accurately reproduce the state of the positioning with respect to the simulation irradiation port 107 using the second moving mechanism 110B.

The neutron capture therapy apparatus 1 may further include the fixation portion 60 that fixes the patient 50. The first moving mechanism 110A and the second moving mechanism 110B may move the fixation portion 60 to move the patient 50, and the transport mechanism 120 may transport the patient 50 together with the fixation portion 60. In this case, a posture of the patient 50 in a case where the positioning thereof is performed with respect to the simulation irradiation port 107 is kept constant from the transport by the transport mechanism 120 to the positioning by the first moving mechanism 110A. Therefore, in the treatment unit 102, the first moving mechanism 110A can accurately reproduce the state of the positioning with respect to the simulation irradiation port 107 using the second moving mechanism 110B.

The present disclosure is not limited to the above embodiment.

For example, in the above embodiment, the patient 50 is fixed to the fixation portion 60. However, the patient 50 does not necessarily have to be fixed. In this case, in a case where the positioning of the patient 50 with respect to the simulation irradiation port 107 is completed, a positional relationship between the vicinity of the affected part of the patient 50 and the second collimator 108 is measured by an image, which is captured by a laser sensor or an imaging device, or the like and is stored in the storage unit 151. Next, in the treatment unit 102, a measurement result in the preparation unit 104 is acquired from the storage unit 151, and the patient 50 is moved by the first moving mechanism 110A such that the positional relationship between the patient 50 and the first collimator 20 is the same as the measurement result. Accordingly, with the first moving mechanism 110A and the second moving mechanism 110B, the positional relationship at the time of positioning with respect to the simulation irradiation port 107 is substantially the same as the positional relationship between the irradiation port 106 and the patient 50 during the irradiation in the treatment unit 102.

In the above embodiment, the neutron ray N is irradiated from the two directions. However, the neutron ray N may be irradiated from one direction or from three or more directions.

The configurations of the moving mechanisms 110A and 110B are not limited to the above embodiments, and any configuration may be employed as appropriate as long as the patient 50 can be positioned.

For example, the layout shown in FIG. 1 is merely an example and may be changed as appropriate.

The storage unit 151 may store at least a part of the control parameters of the second moving mechanism 110B from the start of the positioning to the completion thereof, in addition to the parameter of the second moving mechanism 110B at the time of positioning with respect to the simulation irradiation port 107. The first moving mechanism 110A may perform the positioning of the patient 50, based on the parameters stored in the storage unit 151.

It should be understood that the invention is not limited to the above-described embodiment, but may be modified into various forms on the basis of the spirit of the invention. Additionally, the modifications are included in the scope of the invention.

What is claimed is:

1. A neutron capture therapy apparatus comprising:
a treatment unit including an irradiator that irradiates an irradiation target with a neutron ray;
a simulation unit that simulates adjustment of a position of the irradiation target with respect to the irradiator in the treatment unit;
a first moving mechanism that is capable of moving the irradiation target in the treatment unit;
a second moving mechanism that is capable of moving the irradiation target with respect to the simulation unit; and
a transport mechanism that is capable of transporting the irradiation target from the second moving mechanism to the first moving mechanism,
wherein the second moving mechanism moves the irradiation target such that a position of the irradiation target with respect to the simulation unit is substantially the same as a predetermined position of the irradiation target with respect to the irradiator in the treatment unit, and
the first moving mechanism moves the irradiation target to the predetermined position of the irradiation target with respect to the irradiator in the treatment unit.

2. The neutron capture therapy apparatus according to claim 1,
wherein the irradiator of the treatment unit includes a first collimator, and
the simulation unit includes a second collimator that simulates the first collimator.

3. The neutron capture therapy apparatus according to claim 2,
wherein the first collimator includes an irradiation outlet through which the neutron ray passes, and
the second collimator includes a simulation irradiation outlet, which is a through-hole in a horizontal direction, obtained by simulating the irradiation outlet.

4. The neutron capture therapy apparatus according to claim 3,
wherein the first collimator is attached to an irradiator periphery wall, and
the second collimator is attached to a simulation wall that simulates the irradiator periphery wall.

5. The neutron capture therapy apparatus according to claim 2,
wherein the first moving mechanism includes a base portion that is provided on a floor surface, at a position separated by a predetermined distance from the irradiator, and extends in an up and down direction, and
the second moving mechanism includes a base portion that is provided on the floor surface, at a position separated by a predetermined distance from the simulation unit, and extends in the up and down direction.

6. The neutron capture therapy apparatus according to claim 5,
wherein a position of the base portion of the second moving mechanism with respect to the second collimator is the same as a position of the base portion of the first moving mechanism with respect to the first collimator.

7. The neutron capture therapy apparatus according to claim 1, further comprising:
a storage unit,
wherein the storage unit stores a parameter of the second moving mechanism at a time of positioning with respect to the simulation unit, and
the first moving mechanism performs positioning of the irradiation target based on the parameter stored in the storage unit.

8. The neutron capture therapy apparatus according to claim 7,
wherein the storage unit stores at least a part of control parameters of the second moving mechanism from a positioning start to positioning completion, in addition to the parameter of the second moving mechanism at the time of the positioning with respect to the simulation unit, and
the first moving mechanism performs the positioning of the irradiation target based on the parameters stored in the storage unit.

9. The neutron capture therapy apparatus according to claim 1, further comprising:
a fixation portion that fixes the irradiation target,
wherein the first moving mechanism and the second moving mechanism move the fixation portion to move the irradiation target, and
the transport mechanism transports the irradiation target together with the fixation portion.

10. The neutron capture therapy apparatus according to claim 9,
wherein the transport mechanism includes an arm portion that is expandable and contractible, and a support portion that is provided at a tip part of the arm portion to support the irradiation target and the fixation portion.

11. The neutron capture therapy apparatus according to claim 10.
wherein the transport mechanism extends the arm portion in a state where the support portion supports the fixation portion to transport the irradiation target to the treatment unit.
